(19) United States
(12) Patent Application Publication
(10) Pub. No.: US 2017/0188952 A1
(43) Pub. Date: Jul. 6, 2017

(54) MEDICAL DEVICE AND SYSTEMS THEREOF FOR EVALUATING AND DISPLAYING MEDICAL PARAMETERS OF A PATIENT

(71) Applicant: Stephen H. Broomes, Douglasville, GA (US)

(72) Inventor: Stephen H. Broomes, Douglasville, GA (US)

(21) Appl. No.: 15/313,402

(22) PCT Filed: May 22, 2015
(86) PCT No.: PCT/US15/32168

§ 371 Date: Nov. 22, 2016

Related U.S. Application Data
(60) Provisional application No. 62/002,226, filed on May 23, 2014.

(51) Int. Cl. A61B 5/00; A61B 5/024; A61B 5/083; A61B 8/08; A61B 8/00; A61B 5/0205; A61B 8/02

(52) U.S. Cl. CPC A61B 5/6822; A61B 5/0205; A61B 5/02438; A61B 8/02; A61B 8/0866; A61B 8/4209; A61B 5/0836

(57) ABSTRACT

Provided are devices, systems and methods for measuring, evaluating and displaying medical parameters in a patient. Specifically disclosed is a medical device which comprises a collar which fits on a patients neck and displays two or more medical parameters. Also disclosed is an ultrasound probe and suction cup for monitoring a patient.

### CROSS-REFERENCE TO RELATED APPLICATIONS

This application claims priority to and benefit of U.S. Provisional Patent Application 62/002,226, filed May 23, 2014, which is fully incorporated by reference and made a part hereof.

### TECHNICAL FIELD

The present application relates to devices, systems and methods for measuring, evaluating and displaying medical parameters of a patient.

### BACKGROUND

In modern medical health care facilities (such as doctors' offices, skilled nursing facilities, chronic care facilities, home care facilities, and hospitals), health care personnel use various medical devices to view patient information or provide medical care to a patient. Some medical care devices administer medical care—for example, an intravenous pump that delivers a solution containing a medication into a patient's bloodstream or a ventilator that delivers oxygen to a patient's lungs. Other medical monitoring devices measure and report a patient's physiological status.

Typically, the patient is lying in a bed surrounded by various health care personnel and monitoring/display devices. This is particularly true when the patient is in “code blue” and attempts to resuscitate/revive the patient are underway. This is often complicated by the fact that various health care personnel are communicating various medical parameters audibly, and sometimes simultaneously. This can lead to confusion and miscommunication, and generally provides for a chaotic atmosphere.

Additionally, “code blue” situations are dynamic and the patient's pulse can be there at one moment in time and then rapidly disappear. It is a frequent occurrence that, after the initial return of a spontaneous circulation, the patient can lose their pulse without anyone on the team knowing it because no one on the team is continuously monitoring the pulse. The reason for this is because electrical activity can be present on the cardiac monitor without the heart actually beating. (PEA or pulseless electrical activity) This can lead the code team to believe that the patient has a pulse when they actually don't. This can obviously lead to periods of time when the patient is not being adequately treated. This situation also exists when patients are in other cardiac rhythms including ventricular tachycardia.

Generally, the various medical devices surrounding a patient's bed operate independently of each other and include non-standard wires, tubes, and interfaces. One problem is lack of integration between the medical devices. For example, some medical devices generate information in a proprietary format, which is not compatible with other medical devices from different vendors. In another example, a medical device may produce an analog signal for a patient's vital signs. Because the signal is not digital or recorded, the analog signal must be transcribed onto a piece of paper or else the information is lost. As a result of this lack of integration, health care personnel must pay greater attention to control and monitor many medical devices individually—requiring more personnel to transcribe the data, more time to review the data, and greater potential for lost data and transcription error. Some devices with analog signals may store the data for short periods of time but again, the time must be taken later to review and transcribe the information.

Additionally, many medical devices operate independent of a health care computer system or an electronic medical record (EMR) in which a database of patient medical records is stored. Consequently, health care personnel need to read information from the medical devices and manually enter the information into the health care computer system for storage in the database. In one example, data from medical devices such as glucometers, EKG apparatuses, intravenous (IV) pumps, blood pressure monitoring, ventilators, and respiratory devices are not linked to the EMR. Manual transfer of information from the medical devices to the health care computer system is time-consuming and prone to error.

Verbal and hand-written orders are prone to error, due to confusion about what was said, difficulty interpreting handwriting, and multiple manual steps required to translate the idea into action. Point of care devices and systems used to replace them tend to be time-consuming because of unwieldy software, the small screen geography offered by many devices, and a lack of decision support.

While prior approaches to improving patient care have had limited success in some circumstances, further improvement is needed in light of the aforementioned problems and inefficiencies with point-of-care medical systems. In particular, there exists a need to integrate point-of-care medical systems and to facilitate communication between health care personnel.

### SUMMARY

Provided are devices, systems and methods for display and monitoring of patient medical information, comprising: a collar configured to be worn on the neck of a patient, the collar including a display unit; a processor in operative communication with the display unit, wherein the processor is configured to: receive at least one input signal regarding the patient, wherein the at least one input signal includes pulse information from the patient and information regarding at least one additional patient medical parameter; and to process the received input signal or signals to extract patient medical information; and transmit the patient medical information to the display unit for display and monitoring of the patient medical information.

Also disclosed is a medical device comprising an ultrasound probe, wherein the medical device comprises one or more suction cups configured to secure the ultrasound probe to a patient.

Further disclosed is a method of displaying patient information, comprising: fitting a patient with a collar configured to be worn on the neck, the collar including a display unit; receiving at least one input signal regarding the patient, wherein the at least one input signal includes pulse information from the patient and information regarding at least one additional patient medical parameter; and processing the received input signal or signals to extract patient medical information; transmitting the patient medical information to the display unit; and displaying patient medical information.

Also disclosed is a method of monitoring pulse of a patient, wherein the method comprises the steps of fitting a suction cup on a patient, wherein the suction cup comprises an ultrasound probe; and monitoring the pulse of the patient.

Lastly, disclosed is a clinical care software system comprising a remote computing device in operative communication with a processor, wherein the processor is configured to: receive at least one input signal regarding a patient, wherein the at least one input signal includes pulse information from the patient and information regarding at least one additional patient medical parameter; and to process the received input signal or signals to extract patient medical information; and transmit the patient medical information to the remote computing device, wherein the remote computing device comprises a display unit.

These and other features and advantages of the present invention will become more readily apparent to those skilled in the art upon consideration of the following detailed description and accompanying drawings, which describe both the preferred and alternative embodiments of the present invention.

### BRIEF DESCRIPTION OF THE DRAWINGS

FIG. 1 shows a system comprising a medical device which comprises a collar 100 to be worn by a patient. The collar 100 comprises a display 101 on which various medical parameters 104, 105, 106, 107, and 108 can be displayed. The collar 100 can also comprise a rear neck support 115, allowing the device to fit securely around the patient's neck. The collar 100 can also comprise suction cups 102 which comprise a probe 122 which can monitor the pulse of the patient. Also shown are various other means of measuring medical parameters 118, 119 and 120, which information can be displayed on the display unit 101 of the collar 100.

FIG. 2 shows the computer system 200 for use with the medical device disclosed herein. The computer system 200 includes a main processor 209, and shows that the system can be integrated with a medication component processor 214, as well as a remote computing device 213, via the internet 212 or other networks.

FIG. 3 shows an overview of the system, including cameras 201 and speakers 302. The patient can be in a hospital bed 304 and can be given various medications via IV 305. The patient can wear the medical device 100 disclosed herein.

FIG. 4 shows the medication component 404. The medication component 404 can comprise medication 405 as well as a display/user input feature 406.

FIG. 5 shows various uses of a medical device comprising an ultrasound probe 502 attached to a suction cup 501. FIG. 5A shows the ultrasound probe 502 attached to a patient in the abdominal area. The medical device can communicate with a processor 506 wirelessly or via hard wire 504. The information obtained from the medical device can be displayed on a display unit 503. FIG. 5B shows the ultrasound probe 502 attached to a human head for cranial monitoring. FIG. 5C shows the ultrasound probe 502 attached to a pregnant patient for fetal monitoring. FIG. 5D shows the ultrasound probe 502 attached to a patient's neck, which is the same device 100 as disclosed above in FIG. 1.

### DETAILED DESCRIPTION

The present invention now will be described more fully hereinafter with reference to specific embodiments of the invention. Indeed, the invention can be embodied in many different forms and should not be construed as limited to the embodiments set forth herein; rather, these embodiments are provided so that this disclosure will satisfy applicable legal requirements.

### Definitions

As used in the specification, and in the appended claims, the singular forms “a,” “an,” “the,” include plural referents unless the context clearly dictates otherwise.

The term “comprising” and variations thereof as used herein are used synonymously with the term “including” and variations thereof and are open, non-limiting terms.

As used throughout, by a “subject” or “patient” is meant an individual. The subject may be a vertebrate, more specifically a mammal (e.g., a human, horse, pig, rabbit, dog, sheep, goat, non-human primate, cow, cat, guinea pig or rodent), a fish, a bird or a reptile or an amphibian. The term does not denote a particular age or sex.

### Medical Device, System, and Methods of Use

### Overview

Disclosed herein is a system for monitoring and treating a patient in a hospital setting. or in the field with EMS professionals, in nursing facilities, urgent care centers, assisted living centers, dialysis centers or anywhere that a patient has the potential to be in cardiac arrest. The device and system disclosed herein can also be used in a pediatric setting, and can be scaled accordingly. For example, a patient who is in “code blue” needs to be monitored and treated rapidly. Often, there are multiple health care personnel involved in the treatment, and present in the room at the same time. The system disclosed herein allows for the coordination of various aspects of treatment, monitoring, and medication being administered simultaneously. The system comprises a collar which can be worn, for example by a patient, which displays multiple medical parameters simultaneously. By being able to view various data points in one place, medical staff are able to more quickly assess the needs and progress of a patient.

Furthermore, the system optionally comprises a medication component, so that medication parameters can be integrated with the other medical parameters for easy viewing and access by all of the medical staff present. The medication component allows for direct input of medications given, and can determine (and display) dosage and timing information for recent and future administration of medication.

The system disclosed herein can also include a wireless component. This can allow for medical staff to assess the state of the patient, and to prescribe treatment while not being physically present. It can also allow for the integration of other information, such as the patient's medical history, into the assessment of the patient. The system can also be set up with cameras and other means of remote monitoring to provide further information to the medical staff regarding the state of the patient.

Also disclosed is an ultrasound probe which can be attached to a patient via a suction cup or other means, such as a foam adhesive. This allows for the probe to be stable, and prevents an ultrasound technician, physician or other ACLS assistants from having to continuously hold the ultrasound probe. In particular, the ultrasound probe with suction cups can be used on the carotid artery, femoral artery, or any other readily accessible artery to measure pulse during a code situation.

This suction cup probe is constructed internally with an array of as many small ultrasound probes as technically possible to monitor the patients pulse. These probes are arranged in such a way as to monitor for a pulse in a 360 degree pattern continuously moving from one probe to the next in a pattern that allows for the pulse to be searched for continuously during the application of the collar. In other locations, this technology can be applied to various parts of the body to monitor pulse. Another example is monitoring the pulse on a patient's leg immediately after a cardiac catheterization to assess for the possible complications associated with those procedures such as distal embolization and a pseudoaneurysm.

### System

Disclosed herein is a system for display and monitoring of patient medical information, comprising a collar 100 configured to be worn on the neck of a patient, the collar including a display unit 101; a processor 209 in operative communication with the display unit 101, wherein the processor 209 is configured to receive at least one input signal comprising data 201 regarding the patient, wherein the at least one input signal includes data 201 such as pulse information from the patient and information regarding at least one additional patient medical parameter; and to process the received input signal or signals to extract patient medical information; and transmit the patient medical information to the display unit 101 for display and monitoring of the patient medical information. The processor 209 executes computer-readable instructions. In one aspect, the computer-readable instructions are stored in a memory that is operatively in communication with the processor 209. In one aspect, the processor 209 executes computer-readable instructions to perform the steps of receiving at least one input signal comprising data 201 regarding the patient, wherein the at least one input signal includes data 201 such as pulse information from the patient and information regarding at least one additional patient medical parameter; processing the received input signal or signals to extract patient medical information; and transmitting the patient medical information to the display unit 101 for display and monitoring of the patient medical information.

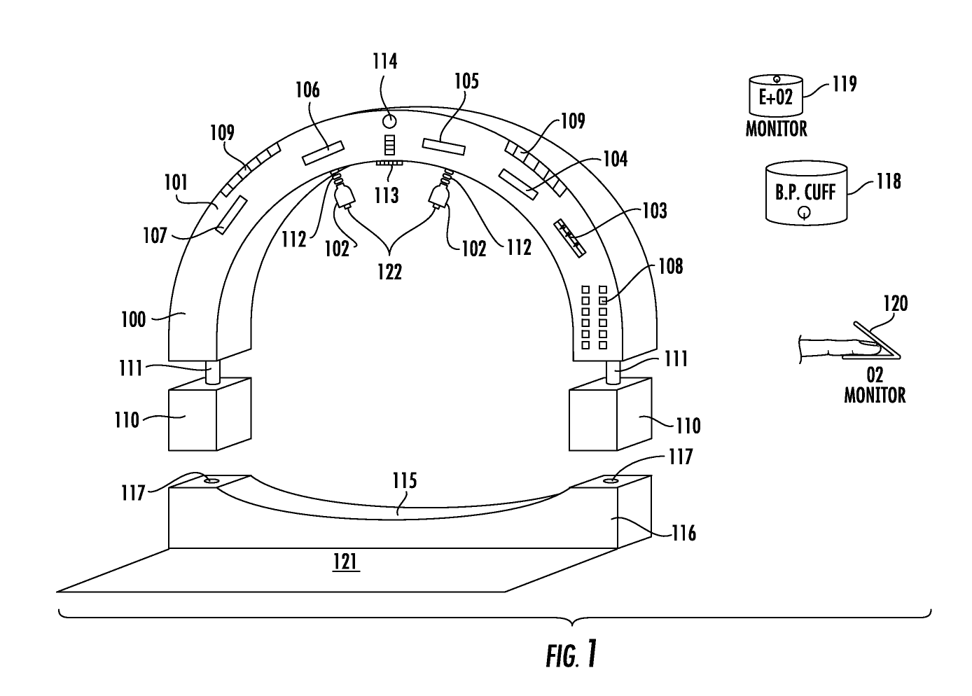

FIG. 1 shows a first sensor 102 can be in operative communication with the processor 209, the sensor configured to sense the pulse information from the patient and to transmit the input signal via the input/output interface 208 regarding the patient including the pulse information to the processor 209. The sensor can be located on the collar 100. For example, the sensor can be a Doppler probe 122, and can be pulsed or continuous wave. The Doppler probe 122 can be located in a suction cup 102 which is in contact with the skin of the patient, and the suction cup 102 can fit onto the patient over the area of the carotid artery or other readily accessible artery in such a manner that the probe can detect pulse. The suction cups 102 can be attached to the collar via springs 112, such that the suction cups 102 are maneuverable in height, thereby allowing for adjustment in where the suction cup 102 fits on the patient, and how tight the point of attachment is to the patient, or how much pressure is being applied to the point of measurement.

The system can also comprise a second sensor which can be in operative communication with the processor 209, the sensor configured to sense the information regarding the at least one additional patient medical parameter and to transmit the data 201 regarding the patent including the at least one additional patient medical parameter to the processor 209. This second sensor can be located on the collar 100, or can be elsewhere on a patient, such as a cuff 118, or on a finger, forehead, ear, chest, or other location. The value related to the at least one additional medical parameter can be a value representative of the patient's end-tidal carbon dioxide, blood pressure, oxygen saturation, pulse oximetry, or wherein the value indicates a drug administration characteristic to the patient. The second sensor can also be configured to measure the patient's temperature or pH, or various other parameters useful in a medical setting.

The system can also receive and display information from various sources, such as laboratory or X-ray results or information from medical professionals not immediately present. For example, the system can be configured to receive messages remotely, such as wirelessly or by SMS.

For example, the second sensor can monitor end-tidal carbon dioxide 119, or capnography. Capnography provides information about CO₂ production, pulmonary (lung) perfusion, alveolar ventilation, respiratory patterns, and elimination of CO₂ from the anesthesia breathing circuit and ventilator. CO₂ sensors are located in line with the patient's breathing circuit (mainstream) or remote from the circuit (sidestream). Sidestream capnometers draw a continuous sample of gas from the respiratory circuit via tiny aspiration pumps for analysis. Mainstream capnometers use specially designed airway adapters with windows, which allow the measurement head to be placed directly in line with the breathing circuit, usually attached to, or close to, the endotracheal tube. Infrared (IR) capnometers are small handheld devices 119. (Donald, Emerg Med J. September 2006; 23(9): 728-730, hereby incorporated by reference in its entirety for its teachings regarding measuring end tidal carbon dioxide). In one example, end tidal CO₂ can be displayed in red, with the color changing to green when adequate chest compressions are given.

Oxygen saturation, another medical parameter, can be measured by using pulse oximetry 120. For example, a processor and a pair of small light-emitting diodes (LEDs) facing a photodiode through a translucent part of the patient's body, usually a fingertip, the forehead, or an earlobe can be used. Such devices for measuring oxygen saturation are known in the art. (Barker S J. “Motion-resistant” pulse oximetry: a comparison of new and old models. Anesth Analg 2002; 95:967-72, incorporated by reference in its entirety for its teaching concerning pulse oximetry).

Blood pressure can also be measured in the patient, such as by a blood pressure cuff 118. Blood pressure can be measured at various intervals, such as every 1, 2, 3, 4, 5, 6, 7, 8, 9, or 10 minutes, or more or less. Alternatively, blood pressure can be monitored continuously. One of skill in the art will appreciate the various ways to measure blood pressure in a patient. (O'Brien, BMJ. Mar. 3, 2001; 322(7285): 531-536, incorporated by reference in its entirety for its teaching concerning measuring blood pressure.)

The system can also comprise a light source 113 useful in tracheal intubations. Because the collar 100 is positioned above the patient's neck in such a manner, it can be useful to have a light source, such as infrared light, to direct a medical professional in intubating the patient. One of skill in the art can appreciate how such a light source can be used in intubation (Sahu J Emerg Trauma Shock. 2009 January-April; 2(1): 51-53, herein incorporated by reference in its entirety for its teaching concerning patient intubation using a light source).

In one example, the system disclosed herein can include a stylet used in intubation which comprises an LED light. The lighted stylet is a device that employs the principle of transillumination to facilitate blind orotracheal intubation (an intubation technique in which the laryngoscopist does not view the glottis).

The collar 100 can be formed of aluminum, plastic, or some combination thereof. The Doppler probe 102 can comprise a Doppler head which can maintain constant contact with the patient, thereby allowing for constant monitoring. In one embodiment, the Doppler head is the only point of contact with the patient. This can be important if the patient is receiving electrical stimulation, so that the patient can be shielded from voltage. In another embodiment, other parts of the collar 100 can be in contact with the patient. When that is the case, the collar can optionally include padding so that it can fit and contour to the neck of the patient.

The display 101 can be configured to display a pulse value of the patient 103 and a value related to the at least one additional patient medical parameter, such as end tidal carbon dioxide 106, oxygen saturation 105, blood pressure 104, medication given 108, or elapsed time that the patient has been monitored 107, for example. One or more of these values can be displayed simultaneously. The values can be displayed in real time, or can be delayed. The various displays can be continuous, if the sensors are continuously monitoring the parameter, or can be displayed in intervals, if the sensors are monitoring the medical parameters in intervals. The display can optionally be formed of LCD, and can sit at an angle so that it can be easily viewed by medical staff. For example, the display can sit at a 45 degree angle with the collar. The display can reside on the top, or either side, of the collar. In one embodiment, the display 101 can be hinged so that the angle at which it is displayed is adjustable.

The system disclosed herein can monitor one, two, three, four, five, six, seven, eight, nine, ten, or more parameters at once, and can have one, two, three, four, five, six, seven, eight, nine, ten, or more sensors which are measuring various medical parameters simultaneously. The sensors which are used with the disclosed system can be chosen based on a patient's individual needs, and can be customized to suit the needs of an individual patient or the attending physicians. As discussed herein, various medical parameters can be measured remotely, such as blood pressure via a cuff or continuous systolic blood pressure. When this is the case, the information can be relayed back to the processor 209 either remotely, such as by Bluetooth™ technology, WiFi (IEEE 802.11n), or via the internet 212.

The system disclosed herein can comprise an audio component. For example, the collar itself can comprise one or more speakers 109. Alternatively, the speakers can be external to the collar 302. For example, the speakers can be elsewhere in the room, and can be in wireless communication with the system, such that audio data is broadcast to the remote speakers. Audio data can comprise pulse information, or other medical parameters which can be broadcast in intervals. In one example, the audio data can comprise an alert that medication needs to be administered to the patient. In another example, audio data can include elapsed time that the patient has been in code blue. For example, the strength of the pulse can be audibly recreated using the LED display.

The system disclosed herein can incorporate a guidance system, for example. This can include a visual or auditory system, or both, which can guide a user through the process of treating the patient. For example, the ACLS protocol can be displayed or broadcast, and a “smart system” can be used to determine what protocol is appropriate for the given situation.

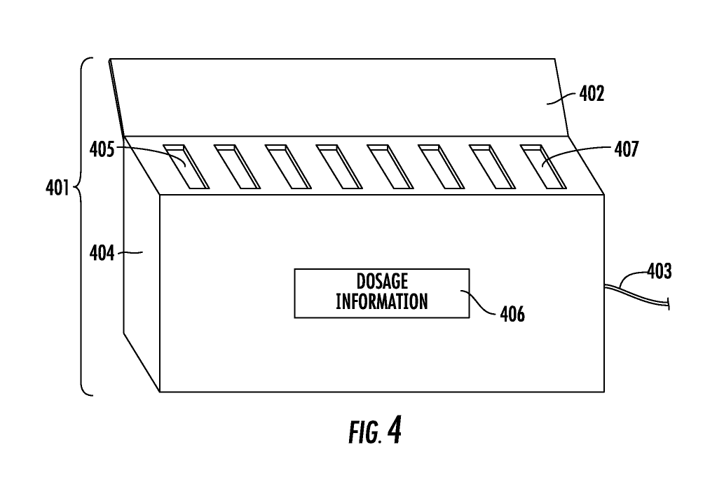

The system disclosed herein can also incorporate a medication component (FIG. 4). For example, the collar can comprise indicator lights 108 which illuminates either when medication needs to be administered to a patient, or when medication has already been given to a patient. The indicator lights can be color-coded such that each medication has a corresponding color, so that the professionals monitoring the patient can easily be apprised of which medications need to be given, or which have already been given. The display can also show any drug administration characteristic. For example, the display can show the drug administered, dose administered, and administration timing. This information can be specific for each drug. This is discussed in more detail herein.

The system disclosed herein can also include an intraosseous IV and the device associated with that. An intraosseous IV is one that is drilled into the tibia. When put in place it provides immediate access to the blood stream. This device can wrap around the lower leg and automatically drill into the tibia. This device can be connected to the medication box disclosed herein, and serve as a way for the box to immediately deliver medications.

The system can also comprise a radio telemetry unit as a part of or separate from the processor 209 for transmitting the patient medical information remotely, such as via the internet 212. The radio telemetry unit can transmit the information to a remote computing device 213, such as a wireless, hand-held communication device. Examples of wireless devices include cell phones and tablets. The computer operating system and associated embodiments are described in more detail herein. The information can also be transmitted via SMS, Bluetooth™ technology, WiFi (IEEE 802.11n), or using other wireless protocols.

The collar 100 can be configured to fit across the front of the patient's neck, and a rear neck support 116 can be configured to contour to the back of the patient's neck. A retractable shelf can be built into the rear neck support, which can allow for adjustment of the patient's head position, to allow for easier intubation when the retractable shelf is retracted. The collar 100 can comprise plungers 110 which are designed to be insertable into receptacles 117 of the rear neck support 116. These plungers 110 can comprise stems which are adjustable in height, thereby comprising an adjustable element 111, so that the collar 100 and rear neck support 116 can be custom-fit to the patient.

The rear neck support can be made of a variety of materials. Examples include, but are not limited to, a rigid but firm material, such as, but not limited to, ethylene-vinyl acetate copolymer (EVA), natural rubbers, polysiloxanes, thermoplastic elastomers including styrene block copolymers, copolyesters, polyurethane block copolymers, polyamide block copolymers, butadiene-acrylonitrile copolymers, butadiene-isobutylene copolymers, polysulfide polymers, and thermoplastic vulcanizates. These exemplary materials are noted because they enable the head and neck support to readily conform to a variety of sizes and shapes, and particularly to a variety of sizes and U-shaped notch 115 which is utilized to receive the head. Polysiloxane rubbers are especially desirable on all aspects of the invention, inasmuch as they include silicones, which are relatively inert materials. The rear neck support can be contoured at an angle 116, which can be particularly useful when the patient's head must be stabilized, or when the neck must be kept at a certain angle in order to intubate the patient.

The rear neck support 116 can be part of a bigger back support 121, and can constructed in such a way as to extend the patient's neck without effort on the part of the health care staff. This can be done by gradually increasing in thickness from the inferior direction of the patient going along the spine to the superior portion of the patient and then abruptly cut off at the base if the neck so that the patients neck naturally extends. It is wide enough to cover the distance between each shoulder and long enough to extend superiorly to the same height as the crown of the patients head. It will extend inferiorly from the crown of the head in a gradually decreasing rate of thickness until it ends. As a result of this configuration a channel is created where the patients head is, and the neck is extended. The additional space superiorly and on either side of the patients head contains holes that can be utilized to hold both intubation equipment and other items that are commonly used during ACLS protocols. For example, clamps to keep the suction catheter used by the person intubating, a variety of blades utilized in various situations during intubation, and any other commonly used items.

The back support 121 can also contain within it a defibrillator gel pad that is inserted ahead of time and that makes direct contact with the patient just inferiorly to the left scapula. This gel pad can have connectivity to the defibrillator via a connector that function as an adapter to the current wiring and allow for seamless and effective defibrillation.

The back support 121 can have a mechanism to elevate itself pneumatically. This can be used on the bottom on the undersurface of the of the backboard so that when the backboard can be raised or lowered by the press of a button. This allows for adjustment, so that patients neck extends or contracts, depending on how much elevation the patient needs. The back support can also comprise an extendable side platform to secure the patient's arm while an IV is inserted, or other measurements or medications given. It can be retractable, so that it can slide out of the way when not in use.

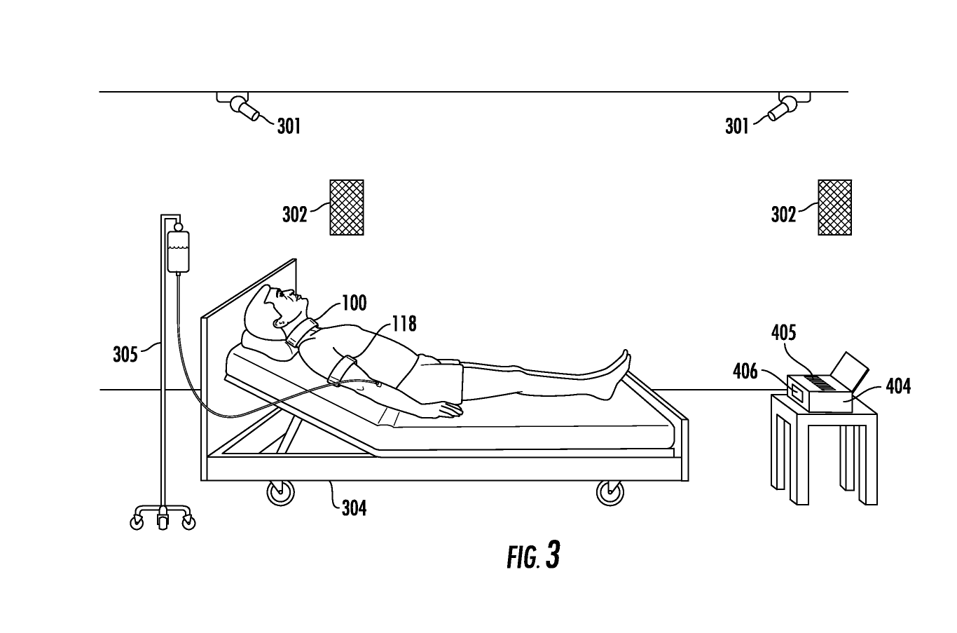

FIG. 3 shows the overall view of the disclosed system in a hospital setting. In this example, the patient wears the collar 100, and is given medication 405 as regulated by the medication component 404. A user, such as a physician or nurse, can input information into the medication component 404 through the interface 406. In one embodiment, information could additionally, or alternatively, be input into the collar 100 itself, or into a remote computing device 213. The system can comprise cameras 301. These can be external to the system, such as cameras mounted in the room so as to record the patient. Video data from the cameras 301 can be sent as data 201 to the processor 209, and can then be transmitted from the processor 209 through the input/output interface 208 to the internet 212, where the video data can be transmitted to a remote computing device 213. This allows for a remote user to access patient information, including images of the patient and their surroundings. For instance, a physician can attend to a patient without being physically present, by using the remote computing device 213 and cameras 301. As discussed herein, speakers 302 can also be external to the collar 100. These speakers can broadcast a variety of useful information, including medication given, medication needed, or the various medical parameters being measured and displayed on the collar 100.

### Medication Component

As seen in FIG. 4, the system can also comprise a medication component 401 with a second processor 214, which can be in communication with the first system processor 209 by being hard wired 403, wirelessly, or via the internet (wired and/or wirelessly) 212. In one aspect, the medication component 401 can be integrated into the collar 100. In other aspects, the medication component 401 may reside separate from the collar 100. The medication component 401 can be, for example, a box 404 with a lid 402. The box can be made of plastic, copper, aluminum, or any other durable material.

The medication component 401 can comprise one or more medications 405 needed for a patient. Examples of such medications 405 can include, but are not limited to, epinephrine, glucagon, vasopressin, atropine, amiodarone, lidocaine, and adenosine, or any other medication recommended in ACLS protocol, or determined to be necessary by an attending physician. The medication component can also include various laboratory tests, or detection methods for determining various parameters of a patient. For example, the medication component can include a quantitative phase-contrast microscopy kit.

The second processor 214 executes computer-readable instructions. In one aspect, the computer-readable instructions are stored in a memory that is operatively in communication with the second processor 214.

The second (medication component) processor 214 can be integral with the medication component 404, and can comprise its own human/machine interface 206 which allows for input into the second processor 214. Alternatively, the medication component processor 214 can be independent of the box 404, and can stand alone as a computer, for example. If this is the case, the medication component 401 can be attached to the medication component processor 214 via wire 403, or can be wireless. The medication component processor can comprise multiple inputs, and can be scalable.

The medication component 404 can also comprise its own display unit 406. Therefore, a user can input dosage and frequency information into the medication component 404. For example, the user can input which medication 405 has been given. The second processor 214 can then automatically determine the needed frequency of the medication, and can interact with the main processor 209 so that indicator lights 108 on the collar 100 can display when medication 405 needs to be given. The second processor 214 can also interact with speakers 109 so that a prompt, such as beep or a voice, can indicate when medication needs to be administered.

The medication display unit 406 can prompt the user for input to determine the dosage or frequency of medication to be given, such as weight, height, race gender, or specific medical condition or history. The medication component 401 can comprise a calculator as part of the second processor 214, so that dosage can be calculated from various physical characteristics or history of the patient. For example, the weight of a patient can be input into the processor 214, and the proper dosage can be calculated for the patient. The proper dosage can then either be administered directly from the medication component 401 through an IV line 407, or can be automatically drawn into a syringe. The medication component can also comprise saline solution, so that a saline flush can be used in an IV or IO (intra-osseous) administration. In another embodiment, the medication component 401 can comprise an intra-osseous drill for immediate use with the patient.

In one embodiment, the medication 405 can be color-coded, so that the label on the bottle or syringe, etc., is the same color as the indicator light 108 on the display unit 101 of the collar 100. This allows for ease of identification of the proper medication, and the color coding can further help medical staff know the dosage schedule for that given medication.

The second processor 214 can also receive specific patient information from existing data 201. For example, any data which is known to exist for a given patient, such as known allergies, medical history, weight, height, etc., can be shared via a bus 210 with the second processor. This allows for medical history to be used concurrently to determine a specific protocol for a given patient. Therefore, it is possible to receive at least one additional input signal, wherein the at least one additional input signal includes information relevant to medication dosage; and to process the received input signal or signals to extract medication dosage information; and transmit the medication dosage information to the display unit either on the collar 100 or on the medication component itself 404, for display and monitoring of the patient medication dosage.

In one example, the medications 405 in the medication component 404 can be pre-mixed. The medication can be in a syringe so that it can be easily administered by a user. Alternatively, the medication can be in a prepared IV, so that it can be easily hooked up to the patient and administered rapidly. In one example, the IV component 407 can be attached to the medication component 401, so that with the touch of a button, the health care professional can administer the proper dosage via IV to the patient.

Information about medication 405 given can be transmitted to the display 101 on the collar 100 or to a remote computing device via a wired connection, wirelessly, or combinations thereof including for example via Bluetooth™, IEEE STd 802.11n, or via the internet 212. One of skill in the art will appreciate that the medication component 401 can be in fluid communication with both the processor 209 of the medical device 100 as well as a remote computing device 213 via a variety of communication methods.

In one embodiment, the medication component 401 can be used as a centralized station to store and charge the collar 100. For example, the medication box 404 can comprise a storage center, so that the collar can be properly stored and protected when not in use. Since the collar 100 can be operated by battery, it can be charged while in the medication component 401, so that it does not need to be plugged in while in use with a patient.

In another embodiment, the medication component can include a cardiac defibrillator. It can also include any other component for measuring any patient parameter, or treating the patient. For example, the medication component, as well as any other needed component other than the collar, can be stored together. This is similar to a “crash cart,” but with the additional components disclosed herein. The crash cart can optionally include the back support described herein, as well as the collar and a charging station for the same. This “crash cart” can also include a method for pressurizing IV fluids, so that saline can be rapidly infused through the Intraosseous IV.

### Ultrasound Probe and Suction Cup

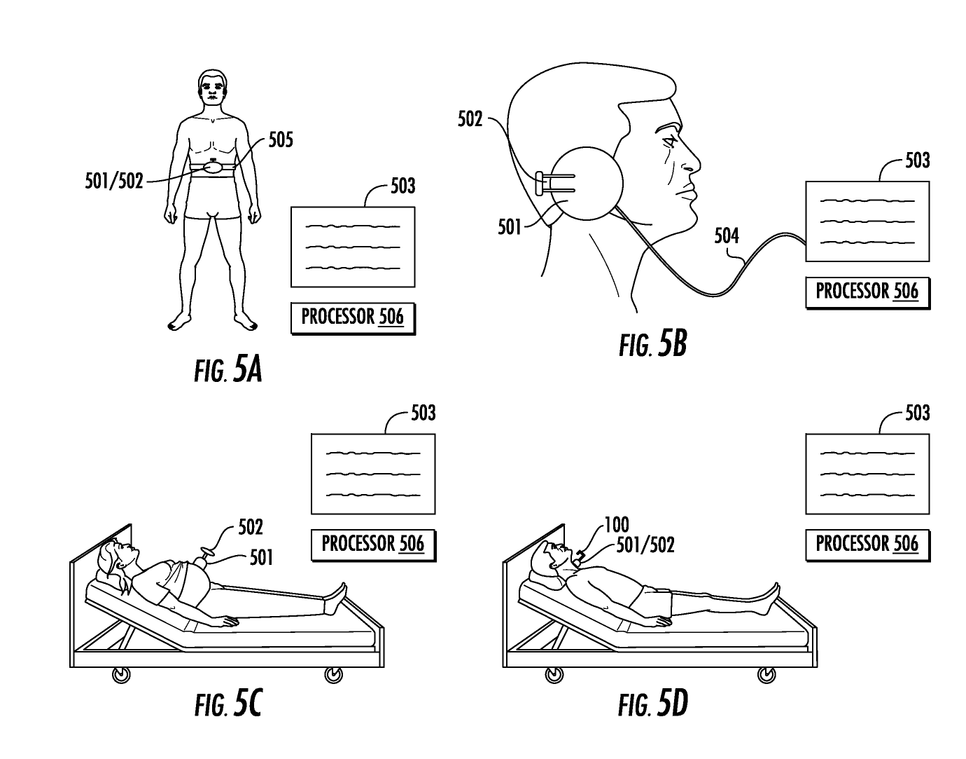

Shown in FIG. 5 is a medical device comprising an ultrasound probe 502, wherein the medical device comprises one or more suction cups 501 configured to secure the ultrasound probe 502 to a patient. The ultrasound probe 502 can be a Doppler probe, and can be continuous or pulsed, or a laser Doppler. One of skill in the art will appreciate all of the various ways an ultrasound probe can be used with a patient. For example, an ultrasound-emitting and receiving device can be used in assessment of: (1) arterial systolic blood pressure; (2) quality of blood flow in arteries and veins (slow flow is less audible than normal flow); (3) direction of blood flow in veins; (4) health of the arterial wall (a triphasic sound indicates good arterial wall elasticity, a biphasic sound indicates moderate arterial wall elasticity and a monophasic sound indicates poor arterial wall elasticity/hardening of arteries); also facilitates calculation of ankle-brachial and toe-brachial pressure indices. Such as device can be external or internal to a patient's body. When attached via suction cup 501, as disclosed herein, the device is external. The suction cup can adhere to the skin of the patient, for example, and can fit over the carotid artery. It can include its own display unit, which can display pulse, for example. Alternatively, the pulse need not be displayed, and can be auditory in nature, or can be displayed on a separate display unit, such as a computer monitor 503.

Pulse need not be detected via ultrasound. For example, pulse can be detected via an arterial line which is a catheter inserted directly into the box, which gives a direct measurement of blood pressure. This can occur in real time, for example. Other examples of pulse detection can be found in U.S. Pat. No. 6,334,850, hereby incorporated by reference in its entirety for its teaching regarding pulse detection.

The ultrasound probe 501 comprising a suction cup 502 can transmit signals wirelessly to a processor and be displayed elsewhere, such as a computer monitor 503, or the display can be integral with the medical device and be displayed directly on the device which is attached to a patient (as disclosed in the collar system above). When the medical device transmits the signal, it can be done wirelessly, or can be wired 504 to connect directly to the processor and/or display unit 503.

The ultrasound probe 502 comprising one or more suction cups 501 can be used in a variety of capacities. For example, in obstetrics and gynecology (FIG. 5C), it can be used for measuring the size of the fetus to determine the due date, determining the position of the fetus to see if it is in the normal head down position or breech, checking the position of the placenta to see if it is improperly developing over the opening to the uterus (cervix), seeing the number of fetuses in the uterus, checking the sex of the baby, checking the fetus's growth rate by making many measurements over time, detecting ectopic pregnancy, determining whether there is an appropriate amount of amniotic fluid cushioning the baby, monitoring the baby during specialized procedures, and seeing tumors of the ovary and breast. The medical device disclosed herein is also useful in cardiology. For example, seeing the inside of the heart to identify abnormal structures or functions, and measuring blood flow through the heart and major blood vessels (FIG. 5B). It is also useful in urological applications, such as measuring blood flow through the kidney (FIG. 5A), seeing kidney stones, and detecting prostate cancer. The suction cup can be further held in place by a strap 505 or by other methods known in the art. Alternatively, it doesn't need a strap, and can be held on simply by the action of the suction cup. It can also be used on the head (FIG. 5B). One of skill in the art will appreciate the various ways ultrasound probes can be used externally. PCT Application WO2011127326, herein incorporated by reference in its entirety, discloses various uses of ultrasound probes.

In one aspect, the probe can “search” for a pulse by rotating in a given space until the pulse (or other parameters which are being searched for) are acquired. This can also be done using multiple probes which rotate about an axis. This rotation aspect can allow for the operator to remain “hands free,” and can considerably improve medical care of the patient.

As described above, the medical device comprising an ultrasound probe 502 can also comprise a processor 506 in operative communication with the display unit 503, wherein the processor is configured to: receive at least one input signal regarding the patient, wherein the at least one input signal includes pulse information from the patient; and to process the received input signal or signals to extract patient medical information; and transmit the patient medical information to the display unit 503 for display and monitoring of the patient medical information. The processor can be configured to receive at least one additional patient medical parameter. For example, as disclosed above, the second sensor can be in operative communication with the processor 506, the sensor configured to sense the information regarding the at least one additional patient medical parameter and to transmit the input signal regarding the patent including the at least one additional patient medical parameter to the processor 506. In one example, the display unit 503 is configured to display the pulse value and the value related to the at least one additional medical parameter simultaneously. The value related to the at least one additional medical parameter can be a value representative of the patient's end-tidal carbon dioxide, blood pressure, oxygen saturation, or wherein the value indicates a drug administration characteristic to the patient.

### Methods

Also disclosed herein are methods of displaying patient information, comprising: fitting a patient with a collar 100 configured to be worn on the neck, the collar including a display unit 101; receiving at least one input signal regarding the patient, wherein the at least one input signal includes pulse information from the patient and information regarding at least one additional patient medical parameter, and transmitting that information via the input/output interface 208; and processing the received input signal or signals to extract patient medical information; transmitting the patient medical information to the display unit 101; and displaying patient medical information.

Also disclosed is a method of monitoring pulse of a patient, wherein the method comprises the steps of: a) fitting a suction cup 501 on a patient, wherein the suction cup 501 comprises an ultrasound probe 502; and b) monitoring the pulse of the patient. As disclosed above, the ultrasound probe 502 can be used to measure a variety of bodily functions, as well as many diseases and conditions. The suction cup can be adhered to the skin of the patient. The adherence to the skin can be enhanced by a variety of methods known in the art, including tape and hydrogel, as well as adhesive foam.

### Software/Hardware System

Functions as described herein can be performed by software, hardware, or a combination of software and hardware. The system can be software, hardware, or a combination of software and hardware. The present methods and systems can be operational with numerous other general purpose or special purpose computing system environments or configurations. Examples of well known computing systems, environments, and/or configurations that can be suitable for use with the system and method comprise, but are not limited to, personal computers, server computers, laptop devices, and multiprocessor systems. Additional examples comprise set top boxes, programmable consumer electronics, network PCs, minicomputers, mainframe computers, distributed computing environments that comprise any of the above systems or devices, and the like.

The processing of the disclosed methods and systems can be performed by software components 202. The disclosed system and method can be described in the general context of computer-executable instructions, such as program modules, being executed by one or more computers or other devices. Generally, program modules comprise computer code, routines, programs, objects, components, data structures, etc. that perform particular tasks or implement particular abstract data types. The disclosed method can also be practiced in grid-based and distributed computing environments where tasks are performed by remote processing devices 213 that are linked through a communications network. In a distributed computing environment, program modules can be located in both local and remote computer storage media including memory storage devices.

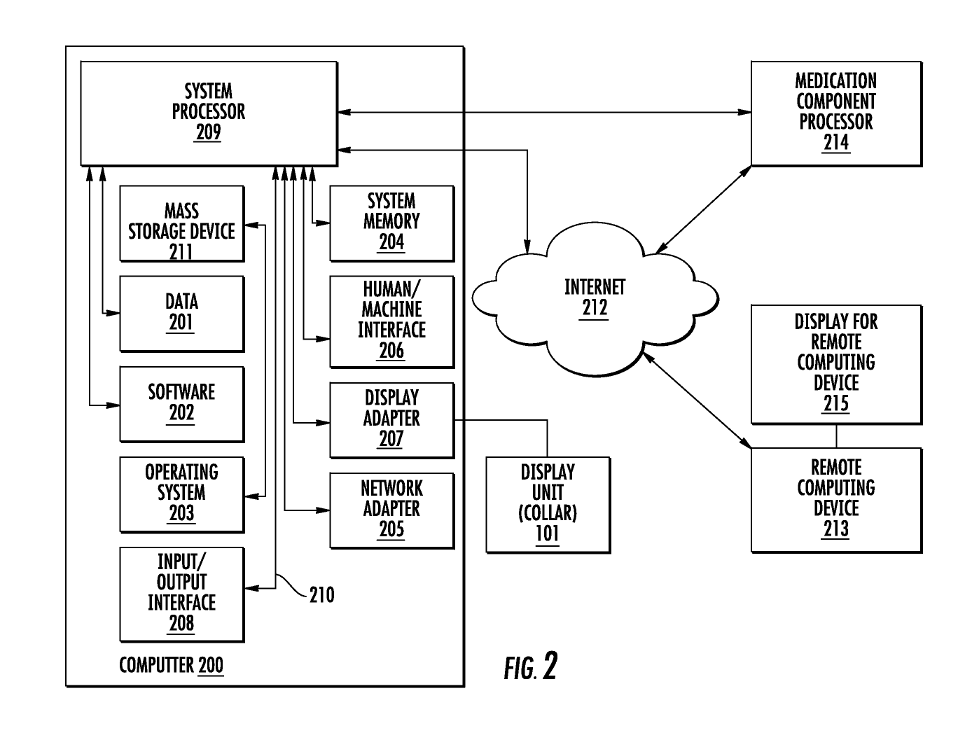

In FIG. 2, the computer system and associated components are shown. One skilled in the art will appreciate that the system and method disclosed herein can be implemented via a general-purpose computing device in the form of a computer 200. The components of the computer can comprise, but are not limited to, one or more processors or processing units 209, a system memory 204, and a system bus 210 that couples various system components including the processor to the system memory 204. In the case of multiple processing units, the system can utilize parallel computing.

The system bus 210 represents one or more of several possible types of bus structures, including a memory bus or memory controller, a peripheral bus, an accelerated graphics port, and a processor or local bus using any of a variety of bus architectures. By way of example, such architectures can comprise an Industry Standard Architecture (ISA) bus, a Micro Channel Architecture (MCA) bus, an Enhanced ISA (EISA) bus, a Video Electronics Standards Association (VESA) local bus, an Accelerated Graphics Port (AGP) bus, and a Peripheral Component Interconnects (PCI) bus also known as a Mezzanine bus. The bus 210, and all buses specified in this description can also be implemented over a wired or wireless network connection and each of the subsystems, including the processor 209, a mass storage device 211, an operating system 203, analysis software 202, received data 201, a network adapter 205, system memory 204, an Input/Output Interface 208, a display adapter 207, a display unit 101 (such as the collar), and a human machine interface 206. One or more of these elements can be contained in one or more remote computing devices at physically separate locations, connected through buses of this form, in effect implementing a fully distributed system.

The computer 200 typically comprises a variety of computer readable media. Exemplary readable media can be any available media that is accessible by the computer 200 and comprises, for example and not meant to be limiting, both volatile and non-volatile media, removable and non-removable media. The system memory 204 comprises computer readable media in the form of volatile memory, such as random access memory (RAM), and/or non-volatile memory, such as read only memory (ROM). The system memory 204 typically contains data such as received data 201 and/or program modules such as operating system 203 and analysis software 202 that are immediately accessible to and/or are presently operated on by the processing unit 209.

In another aspect, the computer 200 can also comprise other removable/nonremovable, volatile/non-volatile computer storage media. By way of example, FIG. 2 illustrates a mass storage device which can provide non-volatile storage of computer code, computer readable instructions, data structures, program modules, and other data for the computer 200. For example and not meant to be limiting, a mass storage device 211 can be a hard disk, a removable magnetic disk, a removable optical disk, magnetic cassettes or other magnetic storage devices, flash memory cards, CD-ROM, digital versatile disks (DVD) or other optical storage, random access memories (RAM), read only memories (ROM), electrically erasable programmable read-only memory (EEPROM), and the like.

Optionally, any number of program modules can be stored on the mass storage device 211, including by way of example, an operating system 203 and analysis software 202. Each of the operating system 203 and analysis software 202 (or some combination thereof) can comprise elements of the programming and the analysis software 202. Received data 201 can also be stored on the mass storage device 211. Received data 201 can be stored in any of one or more databases known in the art. Examples of such databases comprise, DB2®, Microsoft® Access, Microsoft® SQL Server, MAC OS, Oracle®, mySQL, PostgreSQL, and the like. The databases can be centralized or distributed across multiple systems.

In another aspect, the user can enter commands and information into the computer 200 via an input device. Examples of such input devices comprise, but are not limited to, a keyboard, pointing device (e.g., a “mouse”), a microphone, a joystick, a scanner, tactile input devices such as gloves, and other body coverings, and the like. These and other input devices can be connected to the processing unit 209 via a human machine interface 206 that is coupled to the system bus 210, but can be connected by other interface and bus structures, such as a parallel port, game port, an IEEE 1394 Port (also known as a Firewire port), a serial port, or a universal serial bus (USB).

In yet another aspect, a display unit 101 can also be connected to the system bus 210 via an interface, such as a display adapter 207. It is contemplated that the computer 200 can have more than one display adapter 207. The computer 200 can also comprise components such as additional display units, such as a monitor, and a printer (not shown) which can be connected to the computer 200 via Input/Output Interface 208.

The computer 200 can operate in a networked environment using logical connections to one or more remote computing devices 213. By way of example, a remote computing device can be a personal computer, portable computer, a server, a router, a network computer, a cellular phone, a tablet, a peer device or other common network node, and so on. Logical connections between the computer 200 and a remote computing device 213 can be made via a local area network (LAN) and a general wide area network (WAN). Such network connections can be through a network adapter 205. A network adapter 205 can be implemented in both wired and wireless environments. Such networking environments are conventional and commonplace in offices, enterprise-wide computer networks, intranets, and the Internet 212. The remote computing device can have its own display unit 215.

For purposes of illustration, application programs and other executable program components such as the operating system 203 are illustrated herein as discrete blocks, although it is recognized that such programs and components reside at various times in different storage components of the computing device 200, and are executed by the data processor(s) of the computer. An implementation of analysis software 202 can be stored on or transmitted across some form of computer readable media. Computer readable media can be any available media that can be accessed by a computer. By way of example and not meant to be limiting, computer readable media can comprise “computer storage media” and “communications media.” “Computer storage media” comprise volatile and non-volatile, removable and non-removable media implemented in any method or technology for storage of information such as computer readable instructions, data structures, program modules, or other data. Exemplary computer storage media comprises, but is not limited to, RAM, ROM, EEPROM, flash memory or other memory technology, CD-ROM, digital versatile disks (DVD) or other optical storage, magnetic cassettes, magnetic tape, magnetic disk storage or other magnetic storage devices, or any other medium which can be used to store the desired information and which can be accessed by a computer.

The methods and systems can employ Artificial Intelligence techniques such as machine learning and iterative learning. Examples of such techniques include, but are not limited to, expert systems, case based reasoning, Bayesian networks, behavior based AI, neural networks, fuzzy systems, evolutionary computation (e.g. genetic algorithms), swarm intelligence (e.g. ant algorithms), and hybrid intelligent systems (e.g. Expert inference rules generated through a neural network or production rules from statistical learning).

The power source can be electrical, so that it is plugged in, or can be battery-operated. It can have a power switch 109 which can be operated by a user to turn the system on and off. If the source of power is battery, it can be located externally or within the display device. Surface-mounted photodiodes using infrared illumination can also be used with the purpose of providing a continuous power source that does not need to be changed or replaced.

Also disclosed is a clinical care software system comprising a remote computing device or wireless device (213) in operative communication with a processor 209, wherein the processor 209 is configured to: receive at least one input signal from an input/output interface 208 regarding a patient, wherein the at least one input signal includes pulse information from the patient and information regarding at least one additional patient medical parameter; and to process the received input signal or signals to extract patient medical information; and transmit the patient medical information to the remote computing device 213, wherein the personal medical console 213 comprises its own display unit 215. The display unit 215 can be configured to display personal medical information as well. For example, a laptop can be integrated into the device. Such personal medical information can be obtained from electronic health records, or can be stored on the remote computing device, or can be input by the user.

The remote computing device can be associated with the processor 209 of the medical device disclosed herein. The processor 209 can be configured to transmit the medical information to the display unit of the medical device itself 101 as well as the display unit 215 of the remote computing device 213. A historical data store 201 in either the computer 200 of the medical device itself or data in the remote computing device 213, or both, can be configured to store history from one or more of the medical parameters. This data can then be transmitted to a hospital, for example, or used to fill out needed paperwork, such as a code sheet.

The remote computing device 213 can comprise an application which can be used to both receive information and direct treatment of the patient. For example, the application can receive information from the processor 209 of the medical device, and display the various medical parameters on the remote computing device 213. The remote computing device 213 can also comprise the ACLS protocol, and optionally comprise a checklist which allows for medical staff to quickly assess the situation of a given patient, and go through the standard protocol, assessing what needs to be done going forward.

The application of the remote computing device 213 can also prompt questions for the medical staff using the application. For example, questions such as, “Is the patient intubated” can provide critical data to help the medical professional make decisions rapidly. The application can also allow the medical professional using it to control the collar 100 and the medication component 401, so that various medical parameters can be triggered to monitor, or stop monitoring, remotely, or to give or change medication 405 remotely.

The remote computing device 213 can also comprise a component which allows the user to interact with others, and to “share” the information in the application, so that, for example, another physician can review the record and discuss treatment options. For example, the attending physician can contact a specialist, such as a cardiologist, radiologist, or anesthesiologist via the remote computing device 213 (such as a Smart Phone) and share, via the application, the patient's current status. The information in the application can then be discussed, as both parties can view the information simultaneously.

The remote computing device 213 can also be configured so that if the user is remote to the patient, other medical staff who are present can communicate directly with the user to give information. For example, if the remote computing device 213 is a cellular phone, information can be transmitted to the user via text. Or in the case of an application on the remote computing device 213, the medical staff in attendance with the patient can communicate directly through the application. This allows for vital information being processed at the site where the patient is to be transmitted to the user who may be remotely located. Information from the cameras 301 present in the room can also be communicated to the user of the application.

1. A system for display and monitoring of patient medical information, comprising:
a collar configured to be worn on the neck of a patient, the collar including a display unit;
a processor in operative communication with the display unit, wherein the processor execute computer-readable instructions to:
receive at least one input signal regarding the patient, wherein the at least one input signal includes pulse information from the patient and information regarding at least one additional patient medical parameter; and to
process the received input signal or signals to extract patient medical information; and
transmit the patient medical information to the display unit for display and monitoring of the patient medical information.

2. The system of claim 1, further comprising a sensor in operative communication with the processor, wherein the sensor senses the pulse information from the patient and transmits the input signal regarding the patient including the pulse information to the processor.

3. The system of claim 2, wherein the sensor is located on the collar.

4. The system of claim 2 or 3, further comprising a second sensor in operative communication with the processor, wherein the sensor senses the information regarding the at least one additional patient medical parameter and transmits the input signal regarding the patent including the at least one additional patient medical parameter to the processor.

5. The system of claim 4, wherein the second sensor is located on the collar.

6. The system of claim 1, wherein the display of the medical information includes display of a pulse value of the patient and a value related to the at least one additional patient medical parameter.

7. The system of claim 6, wherein the pulse value is displayed simultaneously with the value related to the at least one additional medical parameter.

8. The system of claim 7, wherein the value related to the at least one additional medical parameter is a value representative of the patient's end-tidal carbon dioxide, blood pressure, oxygen saturation, or wherein the value indicates a drug administration characteristic to the patient.

9. The system of claim 8, wherein the drug administration characteristic is selected from the group consisting of drug administered, dose administered, and administration timing.

10. The system of claim 2, wherein the sensor comprises a Doppler probe for measuring pulse.

11. The system of claim 10, wherein the Doppler probe is pulsed.

12. The system of claim 10, wherein the Doppler probe is continuous wave.

13. The system of claim 4, wherein the second sensor comprises infrared light and is used to measure end-tidal carbon dioxide.

14. The system of claim 9, wherein the system comprises indicator lights.

15. The system of claim 14, wherein the system comprises an audio component for audibly broadcasting medical information.

16. The system of claim 4, wherein the second sensor comprises a finger monitor used to measure oxygen saturation.

17. The system of claim 4, wherein the second sensor is located remotely from the collar.

18. The system of claim 17, wherein the second sensor is not located on the neck.

19. The system of claim 1, wherein the collar further comprises one or more suction cups used to secure the collar about the patients neck, and wherein said suction cups comprise Doppler probes.

20. The system of claim 1, wherein the processor further executes computer-readable instructions to display the patient's medical information in real time.

21-89. (canceled)
